(19) United States
(12) Patent Application Publication
(10) Pub. No.: US 2014/0065107 A1
(43) Pub. Date: Mar. 6, 2014

(54) DEVICES, SYSTEMS, AND METHODS FOR MODULATING UTERINE FUNCTION

(71) Applicant: Ohio State Innovation Foundation, Columbus, OH (US)

(72) Inventors: Charles Lockwood, Bexley, OH (US); Edward Funai, New Albany, OH (US); Ali R. Rezai, Columbus, OH (US)

(73) Assignee: Ohio State Innovation Foundation, Columbus, OH (US)

(21) Appl. No.: 14/011,205

(22) Filed: Aug. 27, 2013

Related U.S. Application Data
(60) Provisional application No. 61/693,965, filed on Aug. 28, 2012.

(51) Int. Cl. A61K 38/48; A61K 38/00; A61N 1/36; A61N 2/00; A61N 1/05; A61K 35/12; A61K 31/7088

(52) U.S. Cl. CPC A61K 38/4893; A61K 35/12; A61K 38/00; A61K 31/7088; A61N 2/006; A61N 1/0551; A61N 1/3606
USPC 424/93.7; 424/94.67; 514/17.7; 514/44 R; 600/9; 607/117; 607/62

(57) ABSTRACT

One aspect of the present disclosure relates to a method for modulating uterine function in a mammal. One step of the method includes placing a therapy delivery device into direct contact with a spinal cord target associated with uterine function. Next, the therapy delivery device is activated to deliver a therapy signal to the spinal cord target in an amount and for a time sufficient to effect a change in sympathetic and/or parasympathetic activity in the mammal and thereby modulate uterine function.

### RELATED APPLICATION

This application claims priority to U.S. Provisional Patent Application Ser. No. 61/693,965, filed Aug. 28, 2012, the entirety of which is hereby incorporated by reference for all purposes.

### TECHNICAL FIELD

The present disclosure relates generally to neuromodulatory devices, systems and methods, and more particularly to devices, systems, and methods for modulating uterine function in a mammal.

### BACKGROUND

Preterm labor and preterm delivery during pregnancy represents one of the greatest causes of morbidity for infants in the United States. Generally, pre-term labor is defined as labor that occurs before completion of the 37^{th} week of gestation and the earlier the fetus is born, the more likely that it will be unable to live outside the womb. Pre-term delivery affects approximately one in every eight to ten births, and is the cause of at least 75 percent of the neonatal deaths. Additionally, about 20% of the premature infants that survive preterm delivery die in the first month.

In addition, premature infants that survive face a number of serious health concerns, e.g., low birth weight, breathing problems, and underdeveloped organs and organ systems. As a result, infants born prematurely need to stay in the hospital for extended periods of time and require specialized equipment to allow their health to stabilize. In addition to complications at birth, infants who survive have an increased risk for certain life-long health effects, e.g., cerebral palsy, blindness, lung diseases, learning disabilities and developmental disabilities. Some research also suggests that babies born prematurely are at higher risk for certain health problems as they age.

Although pre-term labor and pre-term delivery often results in death, few medical advances have been made in the medical community to reduce the number of preterm deliveries. Current approaches to the prevention of pre-term birth rely in part on identifying a group of women to whom special attention can be directed. The healthcare providers provide education regarding the signs and symptoms associated with pre-term labor and provide monitoring to identify preterm birth conditions and pre-term labor. For example, health care providers may take steps to stop labor if it starts before the 37^{th} weeks of pregnancy, but such treatment has not been proven efficacious beyond 48 hours. Other examples of conventional treatments for pre-term labor include behavioral modifications, such as bed rest and medications that relax the muscles in the uterus. One possible reason for the paucity of treatment options is the limited information and poor understanding regarding the pathophysiology of pre-term labor and pre-term delivery, which is thought to be multi-factorial.

### SUMMARY

The present disclosure relates generally to neuromodulatory devices, systems and methods, and more particularly to devices, systems, and methods for modulating uterine function in a mammal.

One aspect of the present disclosure relates to a method for modulating uterine function in a mammal. One step of the method includes placing a therapy delivery device into direct contact with a spinal cord target associated with uterine function. Next, the therapy delivery device is activated to deliver a therapy signal to the spinal cord target in an amount and for a time sufficient to effect a change in sympathetic and/or parasympathetic activity in the mammal and thereby modulate uterine function.

Another aspect of the present disclosure relates to a method for modulating uterine function in a mammal. One step of the method includes placing a therapy delivery device, without penetrating the skin of the mammal, into electrical communication with at least one nerve target associated with uterine function. Next, the therapy delivery device is activated to deliver a therapy signal to the at least one nerve target in an amount and for a time sufficient to effect a change in sympathetic and/or parasympathetic activity in the mammal and thereby modulate uterine function.

Another aspect of the present disclosure relates to a method for modulating uterine function in a mammal. One step of the method includes placing a therapy delivery device into direct electrical communication with at least one ANS nerve target associated with uterine function. The ANS nerve target is selected from the group consisting of a hypogastric plexus, an inferior hypogastric plexus, a sacral plexus, a superior mesenteric ganglion, an inferior mesenteric ganglion, a lesser splanchnic nerve, and a sacral splanchnic nerve. Next, the therapy delivery device is activated to deliver a therapy signal to the at least one ANS nerve target in an amount and for a time sufficient to effect a change in sympathetic and/or parasympathetic activity in the mammal and thereby modulate uterine function.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a perspective view of a transcutaneous therapy delivery system secured to a pregnant subject and constructed in accordance with one aspect of the present disclosure;

FIG. 2 is a process flow diagram illustrating a method for modulating uterine function in a mammal according to another aspect of the present disclosure;

FIG. 3 is a process flow diagram illustrating a method for modulating uterine function in a mammal according to another aspect of the present disclosure;

FIG. 4 is a process flow diagram illustrating a method for modulating uterine function in a mammal according to another aspect of the present disclosure; and

FIG. 5 is a process flow diagram illustrating a method for modulating uterine function in a mammal according to another aspect of the present disclosure.

### DETAILED DESCRIPTION

Definitions

Unless defined otherwise, all technical and scientific terms used herein have the same meaning as is commonly understood by one of skill in the art to which the present disclosure pertains.

In the context of the present disclosure, the term “nervous tissue” can refer to any tissues of the autonomic nervous system (ANS) including, but not limited to, neurons, axons, fibers, tracts, nerves, plexus, afferent plexus fibers, efferent plexus fibers, ganglion, pre-ganglionic fibers, post-ganglionic fibers, cervical sympathetic ganglia/ganglion, thoracic sympathetic ganglia/ganglion, afferents, efferents, and combinations thereof.

As used herein, the terms “modulate” or “modulating” can refer to causing a change in neuronal activity, chemistry, and/or metabolism. The change can refer to an increase, decrease, or even a change in a pattern of neuronal activity. The terms may refer to either excitatory or inhibitory stimulation, or a combination thereof, and may be at least electrical, magnetic, optical or chemical, or a combination of two or more of these. The terms “modulate” or “modulating” can also be used to refer to a masking, altering, overriding, or restoring of neuronal activity.

As used herein, the term “intraluminal target site” can refer to a desired anatomical location at which a therapy delivery device may be positioned. The intraluminal target site can comprise a variety of locations, including intraluminal and extraluminal locations innervated by, or in electrical communication with, nervous tissue of the ANS. In one example, an intraluminal target site can comprise an intravascular location in electrical communication with at least one nerve of the ANS. Intraluminal target sites contemplated by the present disclosure are described in further detail below.

As used herein, the term “electrical communication” can refer to the ability of an electric field generated by an electrode or electrode array to be transferred, or to have a neuromodulatory effect, within and/or on at least one nerve, neuron, and/or nervous tissue (e.g., of the ANS).

As used herein, the term “pre-term labor” can refer to the onset of labor any time before the end of a normal gestation period (e.g., at about week 37), and the term “post-term labor” can refer to the onset of labor thereafter.

As used herein, the term “mammal” can refer to any member of the class Mammalia including, without limitation: humans and non-human primates, such as chimpanzees and other apes and monkey species; farm animals or livestock, such as cattle, sheep, pigs, goats and horses; domestic mammals, such as dogs and cats; laboratory animals, such as mice, rats and guinea pigs, and the like. The term does not denote a particular age or sex. Thus, adult and newborn mammals, as well as fetuses, whether male or female, are intended to be included within the scope of this term. Other non-limiting examples of non-human mammals included within the term can include: aardvarks; antelopes; armadillos; badgers; bats; bears; bobcats; buffalo; camels; large cats; cheetahs; civet family; cougars; coyotes; deer; dolphins; donkeys; elephant shrews; elephants; elk; ermine; ferrets; foxes; giraffes; goats; guanacos; hedgehogs; hippopotamuses; hyenas; jaguars; leopards; lions; llamas; lynxes; manatees; marine mammals; marsupials; mink; moles; mongoose family; monotremes; moose; mules; mustelids; ocelots; pine marten; pinnipeds; rabbits; raccoons; pandas; reindeer; caribou; rhinoceroses; rodents; skunks; sloths; solenodons; tapirs; tayras; tigers; vicunas; weasels; whales; wolverine; wolves; yaks; and zebras.

Overview

A brief discussion of the pertinent neurophysiology is provided to assist the reader with understanding certain aspects of the present disclosure. The nervous system is divided into the somatic nervous system and the ANS. In general, the somatic nervous system controls organs under voluntary control (e.g., skeletal muscles) and the ANS controls individual organ function and homeostasis. For the most part, the ANS is not subject to voluntary control. The ANS is also commonly referred to as the visceral or automatic system.

The ANS can be viewed as a “real-time” regulator of physiological functions which extracts features from the environment and, based on that information, allocates an organism's internal resources to perform physiological functions for the benefit of the organism, e.g., responds to environment conditions in a manner that is advantageous to the organism.

The ANS conveys sensory impulses to and from the central nervous system to various structures of the body such as organs and blood vessels, in addition to conveying sensory impulses through reflex arcs. For example, the ANS controls: constriction and dilatation of blood vessels; heart rate; the force of contraction of the heart; contraction and relaxation of smooth muscle in various organs; lungs; stomach; colon; bladder; visual accommodation; and secretions from exocrine and endocrine glands, etc. The ANS does this through a series of nerve fibers, and more specifically through efferent and afferent nerves. The ANS acts through a balance of its two components: the sympathetic nervous system (SNS) and the parasympathetic nervous system (PNS), which are two anatomically and functionally distinct systems. Both of these systems include myelinated preganglionic fibers which make synaptic connections with unmyelinated postganglionic fibers, and it is these fibers which then innervate the effector structure. These synapses usually occur in clusters called ganglia. Most organs are innervated by fibers from both divisions of the ANS, and the influence is usually opposing (e.g., the vagus nerve slows the heart, while the sympathetic nerves increase its rate and contractility), although it may be parallel (e.g., as in the case of the salivary glands). Each of these is briefly reviewed below.

The PNS is the part of the ANS controlling a variety of autonomic functions including, but not limited to, involuntary muscular movement of blood vessels and gut and glandular secretions from eye, salivary glands, bladder, rectum and genital organs. The vagus nerve is part of the PNS. Parasympathetic nerve fibers are contained within the last five cranial nerves and the last three spinal nerves and terminate at parasympathetic ganglia near or in the organ they supply. The actions of the PNS are broadly antagonistic to those of the SNS—lowering blood pressure, slowing heartbeat, stimulating the process of digestion etc. The chief neurotransmitter in the PNS is acetylcholine. Neurons of the parasympathetic nervous system emerge from the brainstem as part of the Cranial nerves III, VII, IX and X (vagus nerve) and also from the sacral region of the spinal cord via Sacral nerves. Because of these origins, the PNS is often referred to as the “craniosacral outflow”.

In the PNS, both pre- and post-ganglionic neurons are cholinergic (i.e., they utilize the neurotransmitter acetylcholine). Unlike adrenaline and noradrenaline, which the body takes around 90 minutes to metabolize, acetylcholine is rapidly broken down after release by the enzyme cholinesterase. As a result the effects are relatively brief in comparison to the SNS.

Each pre-ganglionic parasympathetic neuron synapses with just a few post-ganglionic neurons, which are located near, or in, the effector organ, a muscle or gland. As noted above, the primary neurotransmitter in the PNS is acetylcholine such that acetylcholine is the neurotransmitter at all the pre- and many of the post-ganglionic neurons of the PNS. Some of the post-ganglionic neurons, however, release nitric oxide as their neurotransmitter.

The SNS is the part of the ANS comprising nerve fibers that leave the spinal cord in the thoracic and lumbar regions and supply viscera and blood vessels by way of a chain of sympathetic ganglia running on each side of the spinal column which communicate with the central nervous system via a branch to a corresponding spinal nerve. The SNS controls a variety of autonomic functions including, but not limited to, control of movement and secretions from viscera and monitoring their physiological state, stimulation of the sympathetic system inducing, e.g., the contraction of gut sphincters, heart muscle and the muscle of artery walls, and the relaxation of gut smooth muscle and the circular muscles of the iris. The chief neurotransmitter in the SNS is adrenaline, which is liberated in the heart, visceral muscle, glands and internal vessels, with acetylcholine acting as a neurotransmitter at ganglionic synapses and at sympathetic terminals in skin and skeletal muscles. The actions of the SNS tend to be antagonistic to those of the PNS.

The neurotransmitter released by the post-ganglionic neurons is nonadrenaline (also called norepinephrine). The action of noradrenaline on a particular structure, such as a gland or muscle, is excitatory in some cases and inhibitory in others. At excitatory terminals, ATP may be released along with noradrenaline. Activation of the SNS may be characterized as general because a single pre-ganglionic neuron usually synapses with many post-ganglionic neurons, and the release of adrenaline from the adrenal medulla into the blood ensures that all the cells of the body will be exposed to sympathetic stimulation even if no post-ganglionic neurons reach them directly.

The present disclosure relates generally to neuromodulatory devices, systems and methods, and more particularly to devices, systems, and methods for modulating uterine function in a mammal. As discussed above, the ANS is responsible for regulating the intrinsic function and balance (or homeostasis) of each body organ. The uterus, for example, is innervated by sympathetic and parasympathetic fibers of the ANS. In pregnancy, there is a progressive sympathetic denervation of the uterus, which leaves the myometrium sensitive to circulating norepinephrine (contractile response) and beta-2-adrenergic agents (relaxation response). As described in detail below, the present disclosure advantageously provides devices, systems, and methods for precise and selective control of the ANS to modulate uterine function. By use of such devices, systems and methods, the present disclosure can, in one example, reduce pre-term labor contractions and thereby reduce subsequent pre-term births.

Therapy Delivery Devices

In one aspect, the present disclosure includes various therapy delivery devices (not shown) and related systems configured to modulate uterine function in a mammal. In some instances, therapy delivery devices that may be used to practice the present disclosure may be positioned directly on a target nerve, neuron or nerve structure. In other instances, therapy delivery devices that may be used to practice the present disclosure may be positioned below the skin of a mammal but not directly on a target nerve, neuron or nerve structure. In further instances, therapy delivery devices that may be used to practice the present disclosure may comprise an external device, e.g., positioned in a lumen adjacent a target nerve, neuron or nerve structure. In still further instances, therapy delivery devices used to practice the present disclosure can include an external device, e.g., positioned on the skin of a mammal adjacent a target nerve, neuron or nerve structure. Therapy delivery devices can be temporarily or permanently implanted within, on, or otherwise associated with a mammal in need of uterine function modulation.

Therapy delivery devices of the present disclosure can be configured to deliver various types of therapy signals to a target nerve, neuron or nerve structure. For example, therapy delivery devices of the present disclosure can be configured to deliver only electrical energy, only a pharmacological or biological agent, or a combination thereof. In one example, therapy delivery devices of the present disclosure can comprise at least one electrode and an integral or remote electrical energy generator (not shown), which is in electrical communication with the one or more electrodes and configured to produce one or more electrical signals (or pulses). In another example, therapy delivery devices can include a pharmacological or biological agent reservoir, a pump, and a fluid dispensing mechanism. Non-limiting examples of pharmacological and biological agents can include chemical compounds, drugs, nucleic acids, polypeptides, stem cells, toxins (e.g., botulinum), as well as various energy forms, such as ultrasound, radiofrequency (continuous or pulsed), magnetic waves, cryotherapy, and the like. In yet another example, therapy delivery devices can be configured to deliver magnetic nerve stimulation with desired field focality and depth of penetration. One skilled in the art will appreciate that combinations of the therapy delivery devices above configurations are also included within the scope of the present disclosure.

In some instances, therapy delivery devices can include a stimulator (or inhibitor), such as an electrode, a controller or programmer, and one or more connectors for connecting the stimulating (or inhibiting) device to the controller. In further describing representative electrodes, which are described in the singular, it will be apparent that more than one electrode may be used as part of a therapy delivery device. Accordingly, the description of a representative electrode suitable for use in the therapy delivery devices of the present disclosure is applicable to other electrodes that may be employed.

An electrode can be controllable to provide output signals that may be varied in voltage, frequency, pulse-width, current and intensity. The electrode can also provide both positive and negative current flow from the electrode and/or is capable of stopping current flow from the electrode and/or changing the direction of current flow from the electrode. In some instances, therapy delivery devices can include an electrode that is controllable, i.e., in regards to producing positive and negative current flow from the electrode, stopping current flow from the electrode, changing direction of current flow from the electrode, and the like. In other instances, the electrode has the capacity for variable output, linear output and short pulse-width. In other instances, the electrode can comprise a coil configured to deliver magnetic stimulation.

The electrical energy generator can comprise a battery or generator, such as a pulse generator that is operatively connected to the electrode. For example, the electrical energy generator can include a battery that is rechargeable by inductive coupling. The electrical energy generator may be positioned in any suitable location, such as adjacent the electrode (e.g., implanted adjacent the electrode), or a remote site in or on the mammal's body or away from the mammal's body in a remote location. An electrode may be connected to the remotely positioned electrical energy generator using wires, e.g., which may be implanted at a site remote from the electrode or positioned outside the mammal's body. In one example, implantable electrical energy generators analogous to a cardiac pacemaker may be used.

The electrical energy generator can control the pulse waveform, the signal pulse width, the signal pulse frequency, the signal pulse phase, the signal pulse polarity, the signal pulse amplitude, the signal pulse intensity, the signal pulse duration, and combinations thereof of an electrical signal. The electrical energy generator may be used to convey a variety of currents and voltages to one or more electrodes and thereby modulate the activity of a nerve, neuron, or nerve structure. The electrical energy generator may be used to control numerous electrodes independently or in various combinations as needed to provide stimulation. In some instances, an electrode may be employed that includes its own power source, e.g., which is capable of obtaining sufficient power for operation from surrounding tissues in the mammal's body, or which may be powered by bringing a power source external to the mammal's body into contact with the mammal's skin, or which may include an integral power source.

In other instances, an electrical signal may be constant, varying and/or modulated with respect to the current, voltage, pulse-width, cycle, frequency, amplitude, and so forth. For example, a current may range from about 0.001 to about 1000 microampere (mA) and, more specifically, from about 0.1 to about 100 mA. Similarly, the voltage may range from about 0.1 millivolt to about 25 volts, or about 0.5 to about 4000 Hz, with a pulse-width of about 10 to about 1000 microseconds. The type of stimulation may vary and involve different waveforms known to the skilled artisan. For example, the stimulation may be based on the H waveform found in nerve signals (i.e., Hoffman Reflex). In another example, different forms of interferential stimulation may be used.

To increase activity in a portion of the ANS, for example, voltage or intensity may range from about 1 millivolt to about 1 volt or more, e.g., 0.1 volt to about 50 volts (e.g., from about 0.2 volt to about 20 volts), and the frequency may range from about 1 Hz to about 2500 Hz, e.g., about 1 Hz to about 1000 Hz (e.g., from about 2 Hz to about 100 Hz). In some instances, pure DC voltages may be employed. The pulse-width may range from about 1 microsecond to about 2000 microseconds or more, e.g., from about 10 microseconds to about 2000 microseconds (e.g., from about 15 microseconds to about 1000 microseconds). The electrical signal may be applied for at least about 1 millisecond or more, e.g., about 1 second (e.g., about several seconds). In some instances, stimulation may be applied for as long as about 1 minute or more, e.g., about several minutes or more (e.g., about 30 minutes or more).

To decrease activity in a portion of the ANS, for example, voltage or intensity may range from about 1 millivolt to about 1 volt or more, e.g., 0.1 volt to about 50 volts (e.g., from about 0.2 volt to about 20 volts), and the frequency may range from about 1 Hz to about 2500 Hz, e.g., about 50 Hz to about 2500 Hz. In some instances, pure DC voltages may be employed. The pulse-width may range from about 1 microseconds to about 2000 microseconds or more, e.g., from about 10 microseconds to about 2000 microseconds (e.g., from about 15 microseconds to about 1000 microseconds). The electrical signal may be applied for at least about 1 millisecond or more, e.g., about 1 second (e.g., about several seconds). In some instances, the electrical energy may be applied for as long as about 1 minute or more, e.g., about several minutes or more (e.g., about 30 minutes or more may be used).

The electrode may be mono-polar, bipolar or multi-polar. To minimize the risk of an immune response triggered by the mammal against the therapy delivery device, and also to minimize damage thereto (e.g., corrosion from other biological fluids, etc.), the electrode (and any wires and optional housing materials) can be made of inert materials, such as silicon, metal, plastic and the like. In one example, a therapy delivery device can include a multi-polar electrode having about four exposed contacts (e.g., cylindrical contacts).

A controller or programmer may also be associated with a therapy delivery device. A programmer, for example, can include one or more microprocessors under the control of a suitable software program. Other components of a programmer, such as an analog-to-digital converter, etc., will be apparent to those of skill in the art.

Therapy delivery devices can be pre-programmed with desired stimulation parameters. Stimulation parameters can be controllable so that an electrical signal may be remotely modulated to desired settings without removal of the electrode from its target position. Remote control may be performed, e.g., using conventional telemetry with an implanted electric signal generator and battery, an implanted radiofrequency receiver coupled to an external transmitter, and the like. In some instances, some or all parameters of the electrode may be controllable by the subject, e.g., without supervision by a physician. In other instances, some or all parameters of the electrode may be automatically controllable by a programmer or controller comprising the therapy delivery device.

In one example, the therapy delivery device can be configured for percutaneous placement or implantation. In this instance, the therapy delivery device can comprise one or more implantable electrodes shaped or configured, for example, as a wire, a rod, a filament, a ribbon, a cord, a tube, a formed wire, a flat strip, or a combination thereof. In one example, one or more of the electrodes can comprise a laminotomy electrode array. Laminotomy electrodes, for example, generally have a flat paddle configuration and typically possess a plurality of electrodes (e.g., 2, 3, 4 or more) arranged on the paddle. The arrangement of electrodes on the paddle may be in rows and columns, staggered, spaced, circular, or any other arrangement that will position the electrodes for optimal delivery of electrical energy. The one or more implantable electrodes may be controlled individually, in series, in parallel, or any other manner desired. Once implanted, the implantable electrode(s) may be held in position using any method known to the skilled artisan, such as stitches, epoxy, tape, glue, sutures, or a combination thereof.

In another example, the therapy delivery device can be configured for intravascular or intraluminal placement or implantation. In some instances, a therapy delivery device configured for intravascular or intraluminal placement or implantation can be configured in an identical or similar manner as the expandable electrode disclosed in U.S. patent application Ser. No. 11/641,331 to Greenberg et al. (hereinafter, “the '331 application”).

In yet another example, the therapy delivery device can be configured for transcutaneous neuromodulation. In some instances, transcutaneous neuromodulation can include positioning an electrode on a skin surface so that a therapy signal can be delivered to a target nerve, neuron, or nerve structure. In other instances, transcutaneous neuromodulation can include positioning an electrode, without penetrating the skin of the subject and without necessarily contacting the electrode with the skin surface, so that a therapy signal can be delivered to a target nerve, neuron, or nerve structure. Transcutaneous neuromodulation can additionally include partially transcutaneous methods (e.g., using a fine, needle-like electrode to pierce the epidermis). For example, an electrode may be placed in contact with the uterine muscle directly to modulate the stimulation and contractions. In another example, a surface electrode can be placed into electrical contact with a nerve, neuron, or nerve structure (e.g., of the ANS) associated with uterine function. Generally, an electrical signal used for transcutaneous neuromodulation may be constant, varying and/or modulated with respect to the current, voltage, pulse-width, cycle, frequency, amplitude, and so forth (e.g., the current may be between about 1 to 100 microampere), about 10 V (average), about I to about 1000 Hz, with a pulse-width of about 250 to about 500 microseconds.

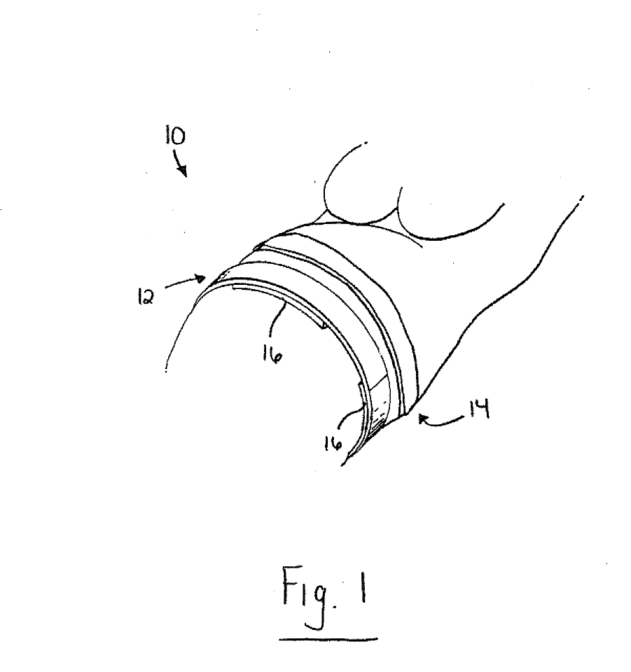

Devices for transcutaneous neuromodulation can have a variety of configurations. In some instances, a transcutaneous therapy delivery device may be configured as a belt or strap having at least one electrode operably attached thereto. Alternatively, a transcutaneous therapy delivery device may be configured as an adhesive patch having at least one electrode operably attached thereto. As shown in FIG. 1, for example, a pregnant subject at increased risk for pre-term labor (as compared to a control) may be prescribed a transcutaneous therapy delivery system 10 by her physician. The transcutaneous therapy delivery system 10 can include adjustable first and second belts or straps 12 and 14 configured for placement about the pregnant subject. The first belt or strap 12 can include at least one electrode 16 configured to deliver a therapy signal to a nerve target associated with uterine contraction and/or the myometrium itself. The second belt or strap 14 can include at least one sensor (e.g., a tocodynamometer) (not shown) configured to detect or monitor uterine function. Alternatively, each of the first and second belts 12 and 14 can include an electrode 16 and a sensor. Each of the straps 12 and 14 can be adjustable so that the electrode(s) 16 can be laid over a nerve target associated with uterine contraction and/or the myometrium itself. Each of the first and second belts or straps 12 and 14 can further include a power source (not shown) for supplying power to the electrode(s) 16 and/or the sensor(s). The power source can be integrally formed with, or wirelessly connected to, each of the first and second belts or straps 12 and 14. In some instances, one or both of the straps 12 and 14 can include a controller (as described above) that is configured, for example, to electronically communicate (e.g., wirelessly) with an electronic device (e.g., a PDA, cell phone, tablet, computer, etc.). In other instances, one or both of the straps 12 and 14 can be attached to, or integrated within, a band of flexible material, such as Spandex, to help maintain the straps about the stomach of the subject. Where the transcutaneous therapy delivery system includes an adhesive patch, it will be appreciated that the patch can further include a sensor configured to detect or monitor uterine function or, alternatively, that the system can include two or more satellite patches, each of which includes a sensor configured to electronically communicate with a planet patch (i.e., having the at least one electrode).

Therapy delivery devices can be part of an open- or closed-loop system. In an open-loop system, for example, a physician or subject may, at any time, manually or by the use of pumps, motorized elements, etc., adjust treatment parameters, such as pulse amplitude, pulse-width, pulse frequency, duty cycle, dosage amount, type of pharmacological or biological agent, etc. Alternatively, in a closed-loop system, treatment parameters (e.g., electrical signals) may be automatically adjusted in response to a sensed physiological parameter or a related symptom indicative of the extent of uterine function. In a closed-loop feedback system, a sensor (not shown) that senses a physiological parameter associated with uterine function can be utilized. More detailed descriptions of sensors that may be employed in a closed-loop system, as well as other examples of sensors and feedback control techniques that may be employed as part of the present disclosure are disclosed in U.S. Pat. No. 5,716,377.

It should be appreciated that incorporating the therapy delivery device as part of a closed-loop system can include placing or implanting a therapy delivery device on or within a mammal at a nerve target, sensing a physiological parameter associated with uterine function, and then activating the therapy delivery device to apply a therapy signal to adjust application of the therapy signal to the nerve target in response to the sensor signal to modulate uterine function. In some instances, such physiological parameters can include any characteristic or function associated with uterine function, such skin temperature, protein concentrations (e.g., fetal fibronectin), uterus and other pressure sensing, electrochemical gradients, hormones (e.g., catecholamines), electrolytes, laboratory values, body temperature, vital signs, changes in cervical length (e.g., as measured by ultrasound), the recorded combination of uterine contractions and the release of fetal fibronectin, or other signs and biomarkers associated with uterine function. In other instances, a therapy delivery device can be operably linked to an external tocodynomometer and/or a myometrial electrode or sensor (e.g., configured to record uterine contractions) to form a closed-loop system.

Methods

Another aspect of the present disclosure includes methods for modulating uterine function in a mammal. More particularly, the present disclosure provides neuromodulatory methods for selective and controlled modulation of myometrial function via the ANS. Both the PNS and the SNS innervate the myometrium, as well as its surrounding structures. Thus, in certain aspects of the present disclosure, the PNS, the SNS, and/or the spinal cord can be selectively stimulated to modulate myometrium function (and thus uterine function) in mammals. Although the methods of the present disclosure are described below in terms of preventing or mitigating pre-term labor, it will be appreciated that the methods can find use in other medical applications as well, such as inducing labor (e.g., in a post-term pregnancy) and/or mitigating pain associated with pregnancy and/or pre-term labor.

In general, methods of the present disclosure can include the steps of: providing a therapy delivery device; placing the therapy delivery device into electrical communication with a nerve target of a mammal that is associated with uterine function; and activating the therapy delivery device to deliver a therapy signal to the spinal cord target in an amount and for a time sufficient to effect a change in sympathetic and/or parasympathetic activity in the mammal and thereby modulate uterine function. The step of placing a therapy delivery device into electrical communication with a nerve target can entail different surgical and/or medical techniques, depending upon the nerve target, for example. In some instances, a therapy delivery device can be surgically placed into electrical communication with a nerve target via a percutaneous or endoscopic route. In other instances, a therapy delivery device can be placed into electrical communication with a nerve target via an intravascular route. In further instances, a therapy delivery device can be placed into electrical communication with a nerve target via a trans-vaginal, trans-cervical, or trans-uterine route. In still further instances, a therapy delivery device can be placed into electrical communication with a nerve target via a transcutaneous approach.

Examples of nerve targets into which a therapy delivery device may be placed in electrical communication with can include, but are not limited to, a spinal nerve, a preganglionic fiber of a spinal nerve, a postganglionic fiber of a spinal nerve, a pelvic nerve, a sympathetic chain ganglion, a thoracic sympathetic chain ganglion, a cervical chain ganglion, a lower cervical chain ganglion, an inferior cervical chain ganglion, a stellate ganglion, a hypogastric plexus, an inferior hypogastric plexus, a sacral plexus, an inferior mesenteric ganglion, a superior mesenteric ganglion, a lesser splanchnic nerve, a sacral nerve, and a spinal cord (e.g., at the level of T10-L1 or S2-S4).

In one example, a therapy delivery device can be placed into electrical communication with a nerve target of a human.

In another example, a therapy delivery device can be placed into electrical communication with a nerve target of a domesticated animal (e.g., a pet) or livestock.

In yet another example, a therapy delivery device can be configured for transcutaneous neuromodulation using magnetic stimulation. A magnetic stimulation device or system can generally include a pulse generator (e.g., a high current pulse generator) and a stimulating coil capable of producing magnetic pulses with desired field strengths. Other components of a magnetic stimulation device can include transformers, capacitors, microprocessors, safety interlocks, electronic switches, and the like. In operation, the discharge current flowing through the stimulating coil can generate the desired magnetic field or lines of force. As the lines of force cut through tissue (e.g., neural tissue), a current is generated in that tissue. If the induced current is of sufficient amplitude and duration such that the cell membrane is depolarized, nervous tissue will be stimulated in the same manner as conventional electrical stimulation. It is therefore worth noting that a magnetic field is simply the means by which an electrical current is generated within the nervous tissue, and that it is the electrical current, and not the magnetic field, which causes the depolarization of the cell membrane and thus stimulation of the target nervous tissue. Thus, in some instances, advantages of magnetic over electrical stimulation can include: reduced or sometimes no pain; access to nervous tissue covered by poorly conductive structures; and stimulation of nervous tissues lying deeper in the body without requiring invasive techniques or very high energy pulses.

After placing the therapy delivery device, the therapy delivery device can be activated to deliver a therapy signal to the nerve target site and thereby modulate uterine function. In some instances, the therapy signal can include an electrical signal capable of electrically modulating the nerve target. In one example, the therapy signal can include an electrical signal capable of electrically modulating at least a portion of the ANS. Electrical modulation of the ANS may affect central motor output, nerve conduction, neurotransmitter release, synaptic transmission, and/or receptor activation. For example, at least a portion of the ANS may be electrically modulated to alter, shift, or change parasympathetic activity from a first state to a second state, where the second state is characterized by an increase or decrease in parasympathetic activity relative to the first state. Alternatively, at least a portion of the ANS may be electrically modulated to alter, shift, or change sympathetic activity from a first state to a second state, where the second state is characterized by an increase or decrease in sympathetic activity relative to the first state.

It will be appreciated that delivering electrical energy, for example, to a target nerve can modulate the ANS in any desirable combination of ways, such as increasing both parasympathetic and sympathetic activity, increasing parasympathetic activity while decreasing sympathetic function, decreasing both parasympathetic and sympathetic activity, and decreasing parasympathetic activity while increasing sympathetic activity.

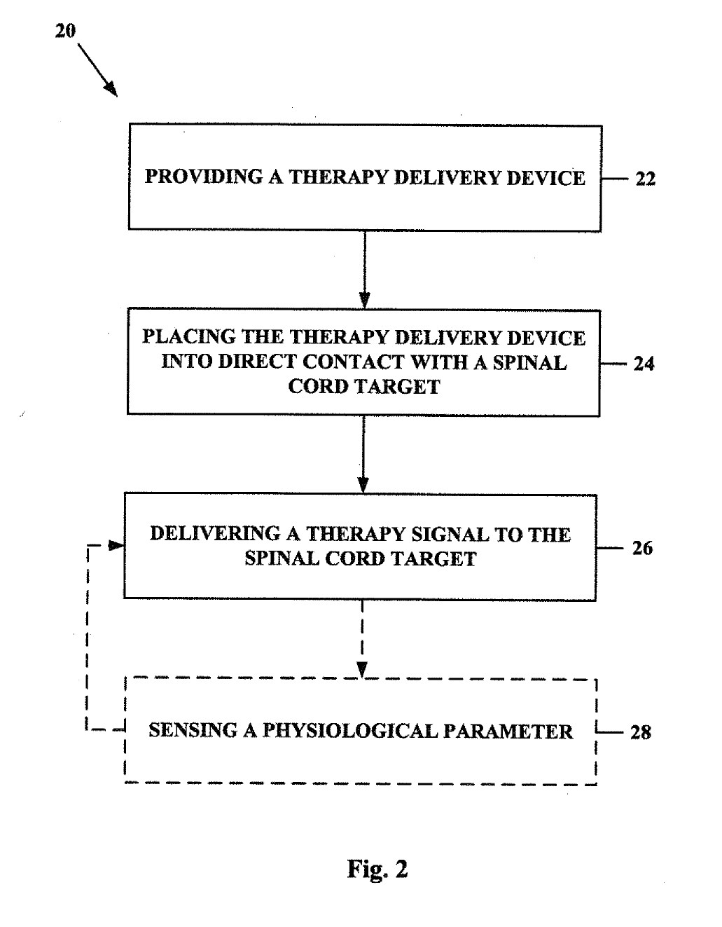

Another aspect of the present disclosure is illustrated in FIG. 2 and includes a method 20 for modulating uterine function in a mammal to prevent or reduce pre-term labor. Generally, the method 20 can entail delivering one or more therapy signals (e.g., electrical energy) to a spinal cord target associated with uterine function. Thus, at Step 22, the method 20 can include providing a therapy delivery device configured for percutaneous placement or implantation. For example, the therapy delivery device can comprise one or more wire or filament-shaped electrodes. Other configurations of the therapy delivery device are described above and known by those skilled in the art.

At Step 24, the therapy delivery device can be percutaneously placed so that one or more electrodes of the therapy delivery device is/are in direct contact with a target region of the spinal cord. The particular target region can be pre-determined (e.g., by a medical professional) based on the medical needs of the mammal. Where the mammal is suffering from pre-term labor, for example, the target spinal cord region can comprise a portion of the spinal cord at the level of T10-L1. Alternatively, the target spinal cord region can comprise a portion of the spinal cord at the level of S2-S4. Additional target spinal regions can include all lumbar and sacral segments of the spinal cord associated with uterine function.

An introducer needle (not shown) may be employed to implant the therapy delivery device on or in the target spinal cord region. The size of the introducer needle may vary depending on the diameter of the therapy delivery device, for example. In some instances, an introducer needle may be a 12-gauge, 14-gauge, 16-gauge, 18-gauge, 20-gauge or 22-gauge needle. It should be understood that other introducer needles may be used as appropriate to the needs and skill level of the practitioner performing the surgical procedure. At least one imaging apparatus, such as a CT scan, MRI apparatus, ultrasound apparatus, fluoroscope, or the like, may be employed to monitor the surgical procedure during localization of the target spinal cord region, e.g., to assist in determining a suitable entry point for the insertion of the therapy delivery device. Once the entry point is determined, the skin overlying the entry point can be shaved and prepared with antiseptic solution. In addition to the local anesthetic, the mammal may be given intravenous sedation and prophylactic antibiotics prior to commencement of the implantation procedure.

The introducer needle can be inserted at the entry point and then advanced to the target spinal cord region. A fluoroscope, for example, may be adjusted as the introducer needle is advanced. Once the introducer needle is suitably positioned, a stylet (not shown) can be withdrawn from the introducer needle. A “test” electrode, if employed, used to test the placement of the introducer needle may then be positioned within a central channel of the needle. If a “test” electrode is not employed, the therapy delivery device that is to be employed may be positioned within the central channel of the needle. The test electrode may then be advanced to the distal tip of the introducer needle to place the test electrode on, in, or proximate the target spinal cord region.

If a “test” electrode is employed to test the placement of the introducer needle and, as such, is different from the therapy delivery device to be employed to modulate the ANS, the “test” electrode may be removed from the introducer needle while the introducer needle is held firmly in place to prevent displacement. The therapy delivery device to be implanted may then be inserted through the central channel of the introducer needle while the introducer needle is held in place. Once the therapy delivery device to be implanted is in position, imaging and electrical stimulation may be employed to verify the correct positioning of the introducer needle and the test electrode. Alternatively, if the electrode used to test the placement of the introducer needle is the therapy delivery device to be implanted, the electrode may be left in the final test position.

Once the implanted therapy delivery device is in place, the end of the device that is outside the skin can be carefully held in place against the skin. The introducer needle may then be slowly removed, leaving the implanted therapy delivery device in place. At this point, if desired, a few small subcutaneous sutures may be placed around the therapy delivery device to hold it in the desired position. A distal end of the therapy delivery device may then be connected to an extension wire or catheter, which is tunneled to the subclavicular area, e.g., or another region that will house the energy source for the implanted therapy delivery device. The device (or devices) used to control or stimulate the therapy delivery device may be surgically implanted in the desired region by procedures known in the art.

Following implantation, the therapy delivery device can be activated at Step 26. The therapy delivery device can be activated to deliver electrical energy, for example, to the target spinal cord region in an amount and for a time sufficient to effect a change in sympathetic and/or parasympathetic activity in the mammal. In one example, electrical energy can be delivered to a therapy delivery device that is implanted at, and in direct contact with, the spinal cord at the level of T10-L1. It may be desirable to do so where the mammal is exhibiting pre-term labor contractions as a result of hyposympathetic tone in the uterus. In this case, electrical energy can be delivered to the therapy delivery device to increase sympathetic activity to a point sufficient to reduce or mitigate the incidence of premature uterine contractions. Alternatively, where unwanted uterine contractions are due to hypersympathetic tone, electrical energy can be delivered to the spinal cord (e.g., at the level of T10-L1) to diminish or reduce sympathetic activity and thereby prevent or mitigate the incidence of pre-term uterine contractions. In another example, electrical energy can be delivered to the spinal cord at the level of S2-S4 to increase parasympathetic activity, thereby causing relaxation of the uterus and thus reducing or eliminating premature uterine contractions in the mammal.

Where the therapy delivery device is configured as a closed-loop system, the method 20 can additionally or optionally include sensing a physiological parameter (discussed above) associated with uterine function (Step 28). A sensor signal can then be generated based on the detected physiological parameter. Next, the therapy delivery device can be activated to adjust application of the therapy signal to the target spinal cord region in response to the sensor signal to modulate uterine function (e.g., to prevent or mitigate pre-term labor).

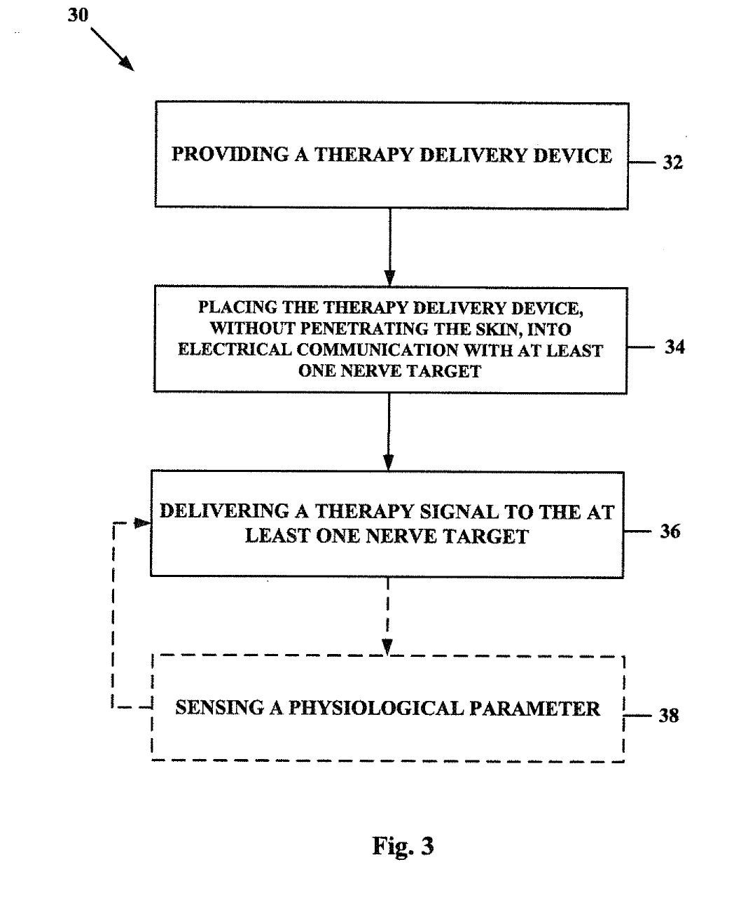

Another aspect of the present disclosure is illustrated in FIG. 3 and includes a method 30 for modulating uterine function in a mammal to prevent or reduce pre-term labor. Generally, the method 30 can entail transcutaneous delivery of one or more therapy signals (e.g., electrical energy) to a nerve target associated with uterine function. Thus, at Step 32, the method 30 can include providing a therapy delivery device configured for placement on the skin of the mammal. Examples of therapy delivery devices configured for transcutaneous delivery of one or more therapy signals are described above.

At Step 34, the therapy delivery device can be positioned about the mammal, without penetrating the skin of the mammal, so that the therapy delivery device is in electrical communication with one or more nerve targets associated with uterine function. Non-limiting examples of nerve targets into which the therapy delivery device can be placed into electrical communication with can include a spinal nerve, a postganglionic fiber of a spinal nerve, a preganglionic fiber of a spinal nerve, a pelvic nerve, a sympathetic chain ganglion, a thoracic sympathetic chain ganglion, a cervical chain ganglion, a lower cervical ganglion, an inferior cervical ganglion, a stellate ganglion, a hypogastric plexus, an inferior hypogastric plexus, a sacral plexus, an inferior mesenteric ganglion, a superior mesenteric ganglion, a lesser splanchnic nerve, and a sacral splanchnic nerve. It will also be appreciated that the therapy delivery device can be positioned about the mammal, without penetrating the skin of the mammal, so that the therapy delivery device is capable of directly modulating uterine muscle function.

After appropriately positioning the therapy delivery device, a therapy signal can be delivered from the therapy delivery device to one or more nerve targets associated with uterine function (Step 36). In one example, a therapy signal (e.g., electrical energy) can be transcutaneously delivered to the inferior hypogastric plexus. It may be desirable to do so where the mammal is exhibiting pre-term labor contractions as a result of hyposympathetic tone in the uterus. In this case, electrical energy can be delivered to the inferior hypogastric plexus to increase sympathetic activity to a point sufficient to reduce or mitigate the incidence of premature uterine contractions. Alternatively, where unwanted uterine contractions are due to hypersympathetic tone, electrical energy (e.g., inhibitory signals) can be delivered to the inferior hypogastric plexus to diminish or reduce sympathetic activity and thereby prevent or mitigate the incidence of pre-term uterine contractions. In another example, electrical energy can be delivered to the sacral plexus to increase parasympathetic activity, thereby causing relaxation of the uterus and reducing or eliminating premature uterine contractions in the mammal.

Where the therapy delivery device is configured as a closed-loop system, the method 30 can additionally or optionally include sensing a physiological parameter (discussed above) associated with uterine function (Step 38). A sensor signal can then be generated based on the detected physiological parameter. Next, the therapy delivery device can be activated to adjust application of the therapy signal to the spinal cord target in response to the sensor signal to modulate uterine function (e.g., to prevent or mitigate pre-term labor).

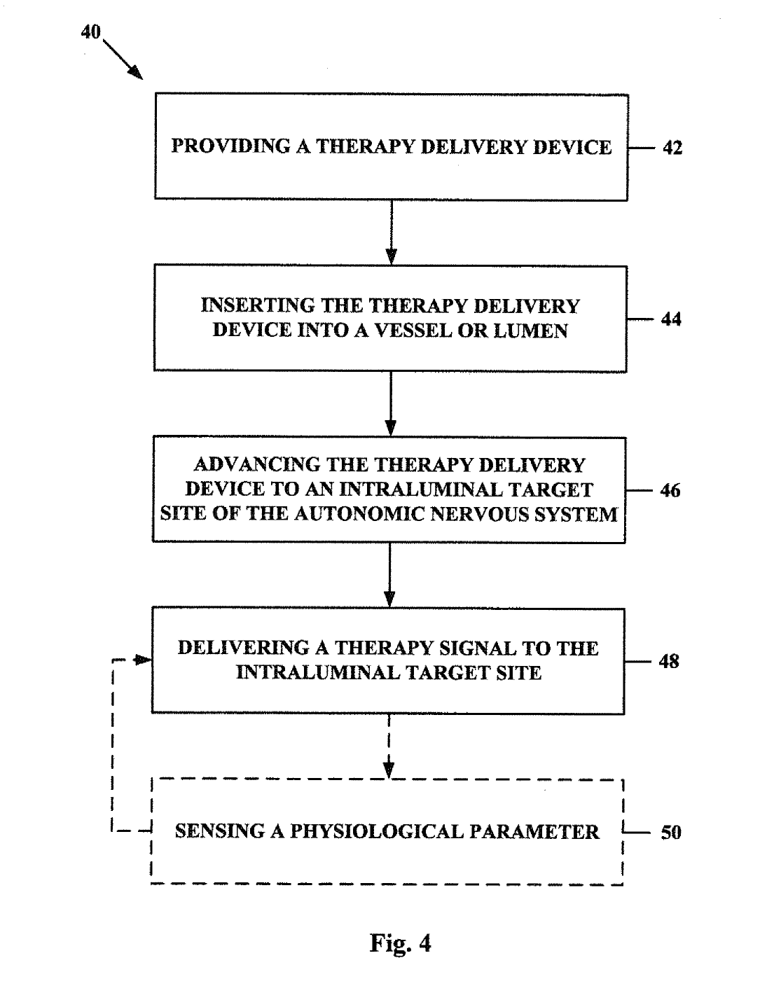

Another aspect of the present disclosure is illustrated in FIG. 4 and includes a method 40 for modulating uterine function in a mammal to prevent or reduce pre-term labor. Generally, the method 40 can entail transvascular or transluminal delivery of one or more therapy signals (e.g., electrical energy) to a nerve target associated with uterine function. Thus, at Step 42, the method 40 can include providing a therapy delivery device configured for transvascular or transluminal insertion and placement within the mammal. In one example, a therapy delivery device configured for intravascular or intraluminal placement in a mammal can include an expandable electrode as disclosed in the '331 application.

At Step 44, the therapy delivery device can be inserted into a vessel or lumen of the mammal. Non-limiting examples of vessel and lumens into which the therapy delivery device can be inserted include an iliac artery (e.g., an internal iliac artery), an ovarian artery, a uterine artery, a vagina and a uterus. The therapy delivery device can be surgically inserted into the vessel or lumen via a percutaneous, transvascular, laparoscopic, or open surgical procedure.

After inserting the therapy delivery device into the vessel or lumen, the therapy delivery device can be advanced (if needed) to an intraluminal target site of the ANS (Step 46). In some instances, advancement of the therapy delivery device can be done under image guidance (e.g., fluoroscopy, CT, MRI, etc.). Intraluminal target sites can include intravascular or intraluminal locations at which the therapy delivery device can be positioned. In some instances, an intraluminal target site can comprise nervous tissue associated with the ANS. For example, an intraluminal target site can include a portion of a vessel wall that is innervated by (or in electrical communication with) a nerve, neuron, and/or nervous tissue of the ANS. Examples of intraluminal target sites can include, without limitation, vascular or luminal sites innervated by and/or in electrical communication with neurons, axons, fibers, tracts, nerves, plexus, afferent plexus fibers, efferent plexus fibers, ganglion, pre-ganglionic fibers, post-ganglionic fibers, cervical sympathetic ganglia/ganglion, thoracic sympathetic ganglia/ganglion, afferents, efferents, a spinal nerve, a preganglionic fiber of a spinal nerve, a pelvic nerve, a sympathetic chain ganglion, a thoracic sympathetic chain ganglion, a cervical chain ganglion, a lower cervical ganglion, an inferior cervical ganglion, a stellate ganglion, a hypogastric plexus, an inferior hypogastric plexus, a sacral plexus, an inferior mesenteric ganglion, a superior mesenteric ganglion, a lesser splanchnic nerve, and a sacral splanchnic nerve.

At Step 48, the therapy delivery device can be activated to deliver a therapy signal to the intraluminal target site in an amount and for a time sufficient to effect a change in sympathetic and/or parasympathetic activity in the mammal. In one example, a therapy delivery device can be inserted into an internal iliac artery and advanced (if needed) to a location substantially adjacent the inferior hypogastric plexus. It may be desirable to do so where the mammal is exhibiting pre-term labor contractions as a result of hyposympathetic tone in the uterus. In this case, electrical energy can be delivered to the inferior hypogastric plexus through a portion of the internal iliac arterial wall to increase sympathetic activity to a point sufficient to reduce or mitigate the incidence of premature uterine contractions. Alternatively, where unwanted uterine contractions are due to hypersympathetic tone, electrical energy (e.g., inhibitory signals) can be delivered to the inferior hypogastric plexus to diminish or reduce sympathetic activity and thereby prevent or mitigate the incidence of pre-term uterine contractions. One skilled in the art will appreciate that a similar intravascular approach may be employed to deliver a therapy signal to the hypogastric plexus and thereby modulate parasympathetic activity in the uterus.

Where the therapy delivery device is configured as a closed-loop system, the method 40 can additionally or optionally include sensing a physiological parameter (discussed above) associated with uterine function (Step 50). A sensor signal can then be generated based on the detected physiological parameter. Next, the therapy delivery device can be activated to adjust application of the therapy signal to the spinal cord target in response to the sensor signal to modulate uterine function (e.g., to prevent or mitigate pre-term labor).

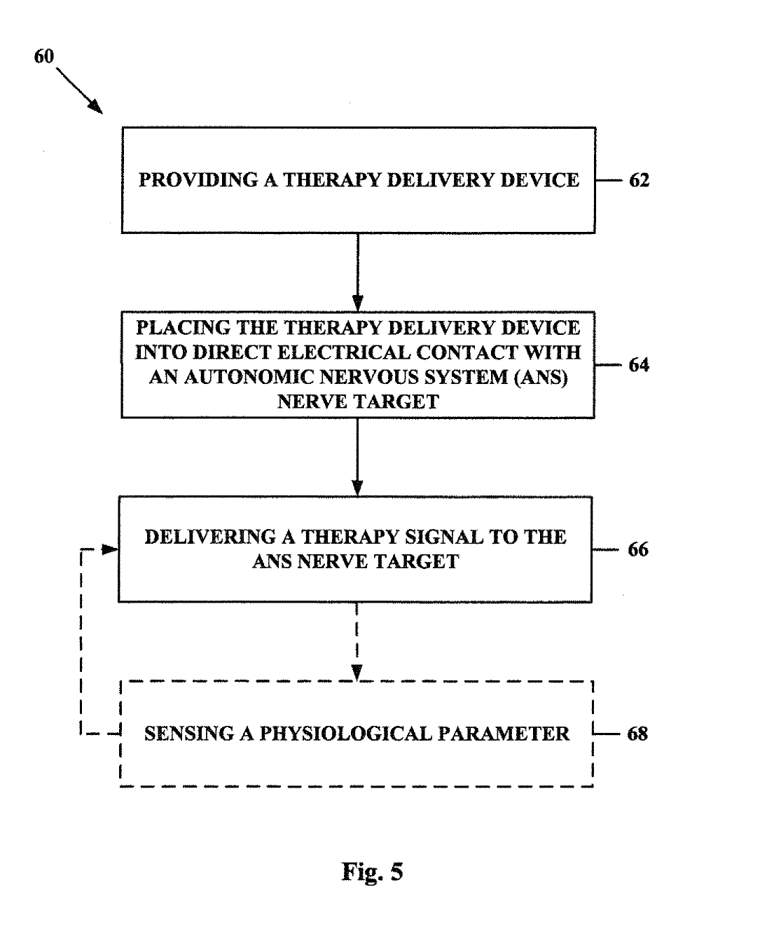

Another aspect of the present disclosure is illustrated in FIG. 5 and includes a method 60 for modulating uterine function in a mammal to prevent or reduce pre-term labor. Generally, the method 60 can entail delivering one or more therapy signals (e.g., electrical energy) to an ANS nerve target associated with uterine function. Thus, at Step 62, the method 60 can include providing a therapy delivery device configured for placement and implantation within the mammal. In one example, the therapy delivery device can comprise an electrode array configured for percutaneous implantation in the mammal.

At Step 64, the therapy delivery device can be placed into direct electrical contact with the ANS nerve target. In some instances, “direct electrical contact” can mean that the therapy delivery device is placed on or in the ANS nerve target. In other instances, “direct electrical contact” can mean that the therapy delivery device is located adjacent (but not in physical contact with) the ANS nerve target such that delivery of a therapy signal can modulate a function, activity, and/or characteristic of the ANS nerve target. Examples of ANS nerve targets can include, but are not limited to, neurons, axons, fibers, tracts, nerves, plexus, afferent plexus fibers, efferent plexus fibers, ganglion, pre-ganglionic fibers, post-ganglionic fibers, cervical sympathetic ganglia/ganglion, thoracic sympathetic ganglia/ganglion, afferents, efferents, a spinal nerve, a preganglionic fiber of a spinal nerve, a pelvic nerve, a sympathetic chain ganglion, a thoracic sympathetic chain ganglion, a cervical chain ganglion, a lower cervical ganglion, an inferior cervical ganglion, a stellate ganglion, a hypogastric plexus, an inferior hypogastric plexus, a sacral plexus, an inferior mesenteric ganglion, a superior mesenteric ganglion, a lesser splanchnic nerve, and a sacral splanchnic nerve.

After placing the therapy delivery device into direct electrical contact with the ANS nerve target, a therapy signal is delivered to the ANS nerve target (Step 66). The therapy signal is delivered in an amount and for a time sufficient to effect a change in sympathetic and/or parasympathetic activity in the mammal. In one example, a therapy signal (e.g., electrical energy) can be delivered to the inferior hypogastric plexus by an electrode or electrode array that is placed directly on or in the inferior hypogastric plexus. It may be desirable to do so where the mammal is exhibiting pre-term labor contractions as a result of hyposympathetic tone in the uterus. In this case, electrical energy can be delivered to the inferior hypogastric plexus to increase sympathetic activity to a point sufficient to reduce or mitigate the incidence of premature uterine contractions. Alternatively, where unwanted uterine contractions are due to hypersympathetic tone, electrical energy (e.g., inhibitory signals) can be delivered to the inferior hypogastric plexus to diminish or reduce sympathetic activity and thereby prevent or mitigate the incidence of pre-term uterine contractions. In another example, electrical energy can be delivered to the sacral plexus (e.g., by placing an electrode or electrode array directly on or in the sacral plexus) to increase parasympathetic activity, which can cause the uterus to relax and thereby reduce or eliminate premature uterine contractions in the mammal.

Where the therapy delivery device is configured as a closed-loop system, the method 60 can additionally or optionally include sensing a physiological parameter (discussed above) associated with uterine function (Step 68). A sensor signal can then be generated based on the detected physiological parameter. Next, the therapy delivery device can be activated to adjust application of the therapy signal to the spinal cord target in response to the sensor signal to modulate uterine function (e.g., to prevent or mitigate pre-term labor).

As mentioned above, it will be appreciated that the devices, systems, and methods of the present disclosure may find use in treating other stages of pregnancy, such as contraction pain, cesarean section, and post-term pregnancies. For example, the present disclosure may be used to treat or reduce pain associated with uterine contractions or cesarean section through the stimulation of certain spinal cord nerves to block pain signals. Additionally, the present disclosure may be used to stimulate labor in post-term pregnancies. For example, one or more therapy delivery devices adapted for electrical communication with a sympathetic nerve target can deliver a therapy signal (or signals) to the target to induce uterine contractions.

From the above description of the present disclosure, those skilled in the art will perceive improvements, changes and modifications. Such improvements, changes, and modifications are within the skill of those in the art and are intended to be covered by the appended claims. All patents, patent applications, and publication cited herein are incorporated by reference in their entirety.

The following is claimed:

1. A method for modulating uterine function in a mammal, the method comprising the steps of:
placing a therapy delivery device into direct contact with a spinal cord target associated with uterine function; and
activating the therapy delivery device to deliver a therapy signal to the spinal cord target in an amount and for a time sufficient to effect a change in sympathetic and/or parasympathetic activity in the mammal and thereby modulate uterine function.

2. The method of claim 1, wherein the mammal is a human.

3. The method of claim 1, wherein the mammal is a domestic animal or livestock.

4. The method of claim 1, wherein the spinal cord target comprises a portion of the spinal cord at the level of T10-L1.

5. The method of claim 4, wherein delivery of the therapy signal effects a change in the sympathetic activity of the mammal.

6. The method of claim 1, wherein the spinal cord target comprises a portion of the spinal cord at the level of S2-S4.

7. The method of claim 6, wherein delivery of the therapy signal effects a change in the parasympathetic activity of the mammal.

8. The method of claim 1, wherein delivery of the therapy signal is effective to reduce or prevent pre-term labor in the mammal.

9. The method of claim 1, wherein delivery of the therapy signal is effective to stimulate labor in a post-term pregnant mammal.

10. The method of claim 8, further comprising the steps of:
sensing a physiological parameter associated with pre-term labor;
generating a sensor signal based on the physiological parameter; and
activating the therapy delivery device to adjust application of the therapy signal to the spinal cord target in response to the sensor signal to prevent or mitigate the pre-term labor.

11. A method for modulating uterine function in a mammal, the method comprising the steps of:
placing a therapy delivery device, without penetrating the skin of the mammal, into electrical communication with at least one nerve target associated with uterine function; and
activating the therapy delivery device to deliver a therapy signal to the at least one nerve target in an amount and for a time sufficient to effect a change in sympathetic and/or parasympathetic activity in the mammal and thereby modulate uterine function.

12. The method of claim 11, further comprising the step of placing the therapy delivery device on the skin of the mammal.

13. The method of claim 11, wherein the nerve target is selected from the group consisting of a spinal nerve, a postganglionic fiber of a spinal nerve, a preganglionic fiber of a spinal nerve, a pelvic nerve, a sympathetic chain ganglion, a thoracic sympathetic chain ganglion, a cervical chain ganglion, a lower cervical ganglion, an inferior cervical ganglion, a stellate ganglion, a hypogastric plexus, an inferior hypogastric plexus, a sacral plexus, an inferior mesenteric ganglion, a superior mesenteric ganglion, a lesser splanchnic nerve, and a sacral splanchnic nerve.

14. A method for modulating uterine function in a mammal, the method comprising the steps of:
placing a therapy delivery device into direct electrical communication with at least one ANS nerve target associated with uterine function; and
activating the therapy delivery device to deliver a therapy signal to the at least one ANS nerve target in an amount and for a time sufficient to effect a change in sympathetic and/or parasympathetic activity in the mammal and thereby modulate uterine function;
wherein the ANS nerve target is selected from the group consisting of a hypogastric plexus, an inferior hypogastric plexus, a sacral plexus, a superior mesenteric ganglion, an inferior mesenteric ganglion, a lesser splanchnic nerve, and a sacral splanchnic nerve.

15. The method of claim 14, wherein delivery of the therapy signal effects a change in the parasympathetic activity of the mammal.

16. The method of claim 14, wherein delivery of the therapy signal is effective to reduce or prevent pre-term labor in the mammal.

17. The method of claim 14, wherein delivery of the therapy signal is effective to stimulate labor in a post-term pregnant mammal.

18. The method of claim 14, further comprising the steps of:
sensing a physiological parameter associated with pre-term labor;
generating a sensor signal based on the physiological parameter; and
activating the therapy delivery device to adjust application of the therapy signal to the at least one ANS nerve target in response to the sensor signal to prevent or mitigate the pre-term labor.
